(19) United States
(12) Patent Application Publication
(10) Pub. No.: US 2025/0251396 A1
(43) Pub. Date: Aug. 7, 2025

(54) MULTIPURPOSE, MULTI-FUNCTIONALIZED LIPID COATED BEADS AND METHODS OF PRODUCTION

(71) Applicant: THE REGENTS OF THE UNIVERSITY OF CALIFORNIA, Oakland, CA (US)

(72) Inventors: Eva de Alba, Merced, CA (US); Christopher J. Randolph, Merced, CA (US)

(73) Assignee: THE REGENTS OF THE UNIVERSITY OF CALIFORNIA, Oakland, CA (US)

(21) Appl. No.: 19/087,544

(22) Filed: Mar. 23, 2025

Related U.S. Application Data
(63) Continuation of application No. PCT/US2023/076052, filed on Oct. 5, 2023.
(60) Provisional application No. 63/413,594, filed on Oct. 5, 2022.

(51) Int. Cl. G01N 33/544; A61K 9/50; A61K 47/69; A61K 49/00; G01N 33/533; G01N 33/543

(52) U.S. Cl. CPC G01N 33/544; A61K 9/5015; A61K 47/6925; A61K 49/0091; G01N 33/533; G01N 33/54353

(57) ABSTRACT

Bead constructs of sizes in the nanometer to micrometer range with a primary functionalization of a lipid membrane with embedded anchor peptides are provided. The anchor peptides may be adapted for a secondary functionalization of active molecules that are bound to the anchor peptides by transpeptidation or similar process. The functionalized bead platform can be adaptable and used in many different applications including biochemical and cellular assays, molecular diagnostics such as protein-protein interactions, protein-DNA interactions, DNA detection, separations, purifications, imaging, and microfluidics.

### CROSS-REFERENCE TO RELATED APPLICATIONS

This application claims priority to, and is a 35 U.S.C. § 111(a) continuation of, PCT international application number PCT/US2023/07605 2filed on Oct. 5, 2023, incorporated herein by reference in its entirety, which claims priority to, and the benefit of, U.S. provisional patent application Ser. No. 63/413,594 filed on Oct. 5, 2022, incorporated herein by reference in its entirety. Priority is claimed to each of the foregoing applications.

The above-referenced PCT international application was published as PCT International Publication No. WO 2024/077122 A1 on Apr. 11, 2024, which publication is incorporated herein by reference in its entirety.

### STATEMENT REGARDING FEDERALLY SPONSORED RESEARCH OR DEVELOPMENT

This invention was made with Government support under Grant No. HRD-2112675 awarded by the National Science Foundation (NSF). The Government has certain rights in the invention.

### INCORPORATION-BY-REFERENCE OF SEQUENCE LISTING

This application includes a sequence listing in a file entitled “UC-2022-910-2-US-seq-listing.xml” created on Oct. 5, 2023 and having a 9 KB file size. The sequence listing is submitted electronically through Patent Center and is incorporated herein by reference in its entirety.

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

This technology pertains generally to constructs and methods for primary and secondary functionalization of beads with membrane-bound biologics (primary functionalization) that are adaptable to be additionally functionalized with other biologics (secondary functionalization) for scientific and engineering applications. The processes preferably create functionalized beads with membrane coatings and secondary functionalization by transpeptidation.

### 2. Background

Beads with sizes in the nanometer to micrometer range are commonly used for multiple scientific and engineering applications including biochemical and cellular assays, molecular diagnostics (e.g., protein-protein interactions, protein-DNA interactions, DNA detection), separations, purifications, imaging, and microfluidics. These diagnostics are useful in the characterization of physiological conditions or the diagnosis of certain disease states. Microarrays, for example, are powerful tools for the analysis of biomolecule interactions that generally operate by immobilizing a set of biomolecules and probing the immobilized biomolecules with potential targets and observing binding.

Commercial beads of various compositions (plastics, glass, magnetic materials, cross-linked materials) and sizes that are functionalized with a variety of different groups are presently available. These groups include different types of biologics (proteins/peptides and nucleic acids) such as antibodies, collagen, protein L, protein G, protein BSA, streptavidin, biotin, neutravidin, and fluorophores, for example. An example of binding assay using functionalized beads known in the art is shown in FIG. 1.

However, functionalized beads like those shown in FIG. 1 are limited in the nature of the functionalization that can be made to the surfaces of the beads. For example, a hydrophobic lipid environment can be essential for and/or promote and/or enhance the specific functions of the groups that are attached to the beads.

Therefore, a need exists for improved functionalized constructs and systems and methods for producing them that expand the potential applications of conventional non-lipid coated beads because of the capability of using biologics with functions that depend on the presence of a lipid environment. There is also a need for improved processes and methods for forming materials that will address these limitations.

### BRIEF SUMMARY

The present technology provides functionalized bead designs and processes of coating beads of various sizes (nanometer to micrometer range) that are made of different materials (e.g., plastics, glass, magnetic materials, cross-linked materials) with lipids or lipid layers with embedded functional molecules (e.g., peptide-derivatives or other molecules) as a primary functionalization. The embedded molecules are designed for a subsequent, secondary functionalization with other molecules (e.g., peptides, proteins, and their derivatives) using an enzymatic reaction or other type of reaction for creating interactions or linkages.

Commercial micrometer-sized beads may be membrane coated and simultaneously functionalized with a hydrophobic biologic that, based on its design, is linked for secondary functionalization via transpeptidation with a sortase to another water-soluble biologic. The protocol only requires basic biochemistry techniques and basic equipment in order to functionalize beads with biologics whose function depends on or is related to their insertion into a membrane, or with biologics that require translational movement around the bead to function.

Beads functionalized with a biologic embedded in a lipid bilayer (different lipids and lipid mixtures can be used) can have potential applications in many different fields for which the presence of lipids is essential or beneficial for the function of the biologic (e.g., membrane binding proteins). In addition, the inherent fluidity of the lipid bilayer may be critical or beneficial for applications that require functional moieties to be mobile (e.g., molecular clustering, multivalent adhesion).

The functionalized beads can be applied as sensors, such as optical traps, for the study of the function of peptides and proteins that are anchored to a biological membrane.

With respect to liposome functionalization embodiments, the advantages of the method are: 1) demonstrated possibility to embed trans-membrane peptides simultaneously to a bead coating with the modified membrane; 2) easy separation of product by simple centrifugation; 3) the demonstrated possibility to link other biologics for secondary functionalization of the beads by transpeptidation of the membrane-inserted peptide; and 4) easy separation of the product by simple centrifugation of the membrane-functionalized coated beads.

With respect to bead coating, the advantages of the method are: 1) the demonstrated possibility of embedding trans-membrane peptides simultaneously to a bead coating with the modified membrane; 2) Easy separation of primary coated beads by simple centrifugation; 3) demonstrated possibility to link other biologics for secondary functionalization of the beads by transpeptidation of one or more membrane-inserted peptide types; 4) easy separation of the product by simple centrifugation of the membrane-functionalized coated beads. Importantly, coating beads with functionalized membranes opens the door to applications that require fluidity and translational movement for proper or enhanced function.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE DRAWINGS

The technology described herein will be more fully understood by reference to the following drawings which are for illustrative purposes only:

FIG. 1 is a schematic diagram of a binding assay using functionalized beads that is representative of what is known in the art.

FIG. 2 is a schematic diagram of a binding assay using functionalized lipid-coated beads according to one embodiment of the technology.

FIG. 3 is a schematic representation of simplified protocol for transmembrane peptide insertion into synthetic membrane coating beads. Liposome formation with transmembrane peptide inserted in the bilayer is followed by bead coating with the peptide embedded synthetic membrane.

FIG. 4 is a schematic representation of secondary functionalization with sortase A to create fluorescent beads according to one embodiment of the technology.

FIG. 5 is a schematic representation of secondary functionalization for DNA sensing according to one embodiment of the technology.

FIG. 6 is a schematic diagram verifying the insertion of a transmembrane peptide in membrane-coated beads and transpeptidation with a fluorescent-labeled peptide. Labeling of a short peptide with Alexa488 by maleimide reaction is followed by a sortase-mediated transpeptidation of inserted transmembrane peptide and Alexa488-labeled peptide.

### DETAILED DESCRIPTION

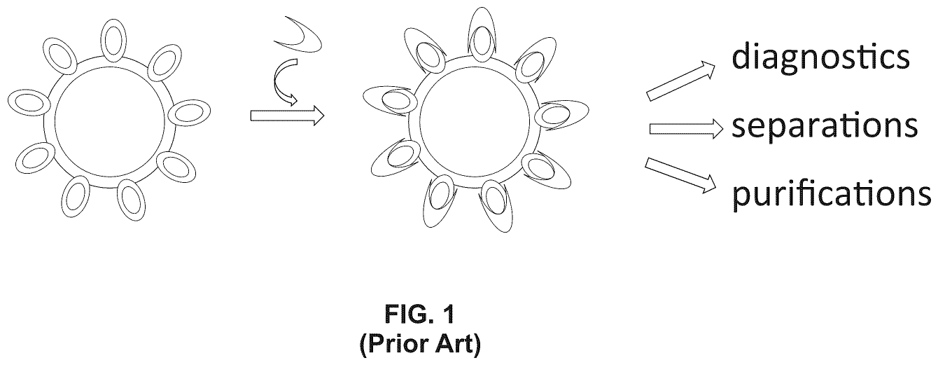

Referring more specifically to the drawings, for illustrative purposes, supramolecular nanocomposite compositions, systems and methods of fabrication and use are generally shown. Several embodiments of the technology are described generally in FIG. 1 to FIG. 6 to illustrate the characteristics and functionality of the compositions, systems, materials and methods. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology.

In general terms, the technology described in this disclosure is a system and applied methods for the primary and secondary functionalization of beads using lipid bilayers and transpeptidation. More specifically, several embodiments of functionalized lipid-coated bead constructs and fabrication methods are described to illustrate the technology. Although a specific construct architecture is used to illustrate the system and methods, other structures and adaptations can be used to achieve the desired functionality of membrane-functionalized lipid-coated beads with primary and secondary functionalization.

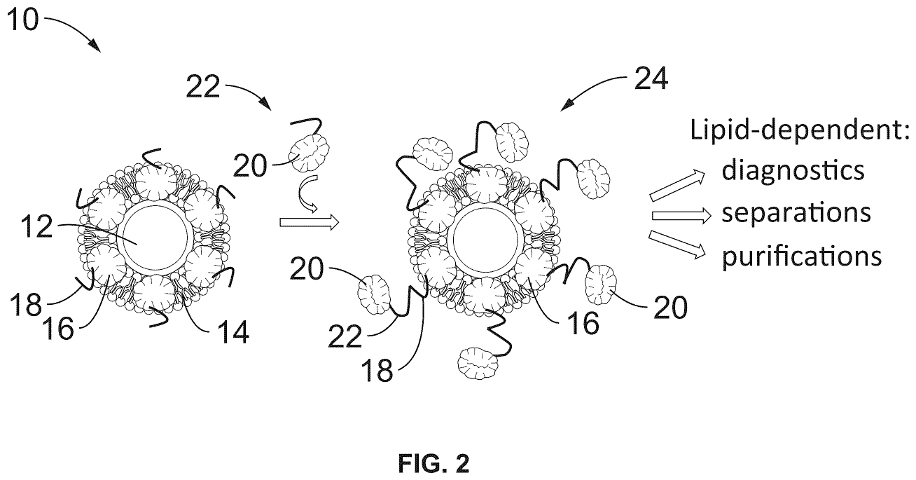

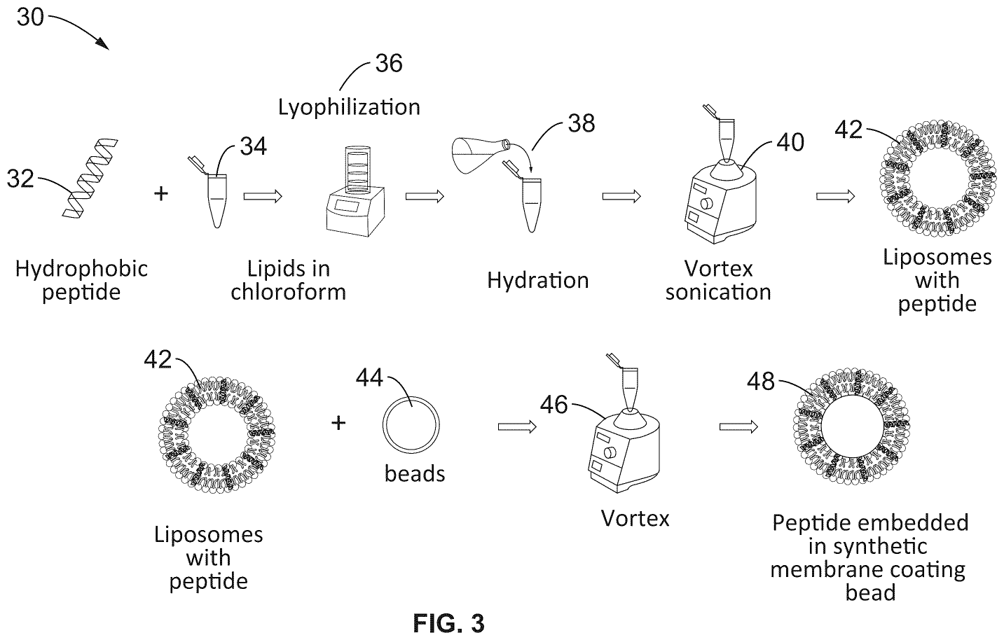

Turning now to FIG. 2 and FIG. 3, a basic design and process for producing a construct of a combination of a coated bead with a functionalized lipid bilayer and secondary functionalization via transpeptidation are shown schematically. The generic construct 10 shown in FIG. 2 has a core 12 that is preferably spherical. Although a generally spherical core 12 is preferred, other three-dimensional shapes, such cylindrical or particulate etc., may also be used. Uniformly sized beads made of plastics, glass, magnetic materials, metals and polymer cross-linked materials are preferred for the core 12 of the construct 10.

The outer surface of the core 12 is preferably smooth and capable of accommodating a layer or bi-layer of lipids 14 that encapsulates the core 12. The lipid layer 14 can be formed with a single type of lipid or it can be formed of different types of lipids or modified lipids in a lipid mixture on the surface of core 12.

Incorporated in the lipid layer 14 are peptides or protein fragments 16 with a hydrophobic end that is incorporated within the lipid layer 16 and a hydrophilic or neutral end that may extend outwardly from the surface of the lipid layer 14. The hydrophilic or neutral end of the peptide 16 is configured to bind to a corresponding primary functional molecule 20 through a chemical linkage 22 such as a chemical bond. This anchor peptide 16 provides a binding site 18 for the linkage 22 of the primary functional molecule 20.

One preferred method 30 for producing a peptide embedded lipid membrane coated bead 48 is shown in FIG. 3. In this embodiment, one or more types of hydrophobic peptides 32 are mixed with one or more types of lipids 34 in the presence of chloroform to form a homogeneous solution. The lipid-peptide mixture solution is then freeze-dried 36 and subsequently hydrated 38 to produce a lipid-peptide resuspension. The re-suspended mixture is then sonicated 40 to produce liposomes or vesicles 42 with embedded peptides. The formed vesicles 42 are centrifuged (16,973 g for 1 h) and the supernatant is mixed with beads 44 and sonicated 46 on low vortex settings for about 16 hours. This mixture is centrifuged (900 g for 1 minute) to pellet the peptide embedded membrane-coated beads 48, which are then ready for use.

The base lipid-coated core 12 with open peptides 16 with binding sites 18 can then be functionalized with a variety of different molecules 20 that provide a selected secondary functionalization for the construct 10. The molecules 16 embedded in the lipid layers are preferably designed for subsequent, secondary functionalization with other molecules, such as peptides, proteins, and their derivatives, using an enzymatic reaction, or other types of reactions for creating interactions or linkages 22.

The selection of the primary functional molecule 20 may also be influenced by a desired secondary functionality such as assay or probe binding protein or nucleic acid fragment.

Secondary functionalization can include any molecule that is capable of embedding in the lipid layer, binding to a selected embedded peptide, or engineered to specifically link to a lipid-embedded peptide. These secondary molecules 20 are also selected based on possessing a desired biological or chemical activity.

These groups typically include different types of biologics such as proteins/peptides and nucleic acids. Target capturing molecules such as antibodies, collagen, protein L, protein G, protein BSA, streptavidin, biotin, neutravidin, and fluorophores, are also suitable for secondary functionalization, for example.

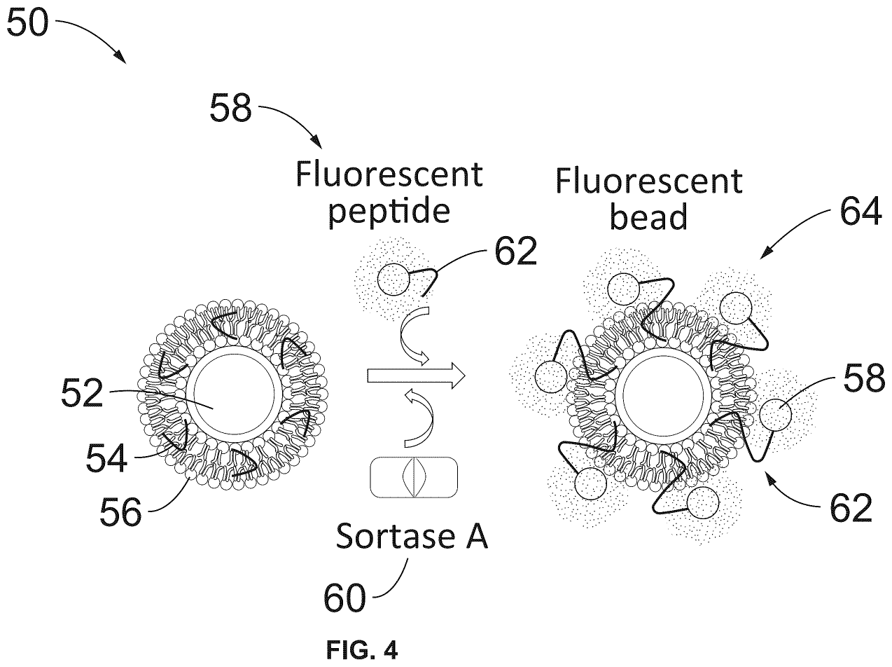

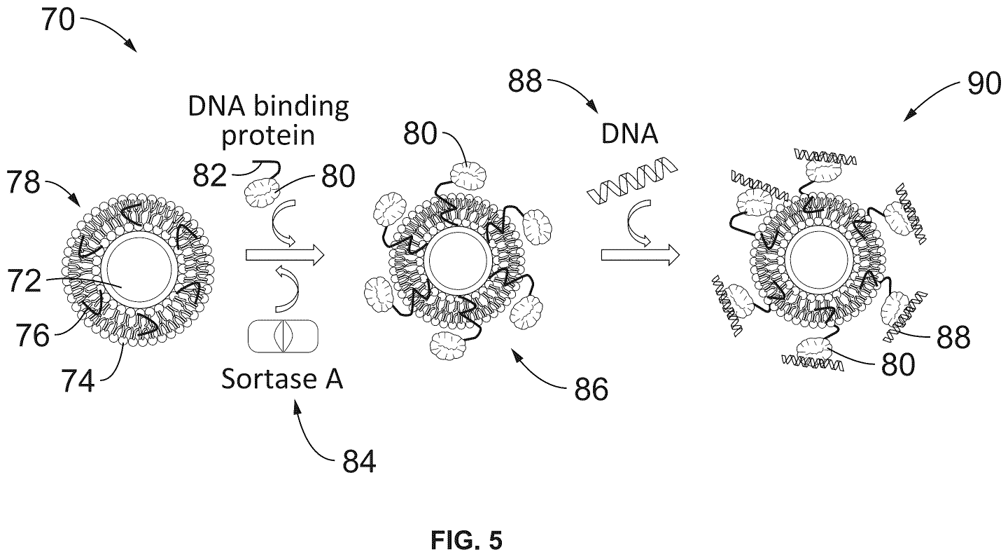

In one embodiment, for example, the primary functional molecule 20 is a peptide that is configured to couple to a fluorescent molecule such as illustrated in FIG. 4. In another embodiment, the primary functional molecule 20 is a DNA or RNA binding protein such as illustrated in FIG. 5. In another embodiment, a fluorescent molecule is attached to the secondary functionalization material.

The broad capacity to control the primary functionalization of the lipid bilayer bead coating and secondary functionalization is an important advantage of the construct and methods. The methods also demonstrate the capability of embedding trans-membrane peptides simultaneously to a bead coating with the modified membrane.

Another advantage of the methods is the capability of linking other biologics for secondary functionalization of the beads by transpeptidation of the membrane-inserted peptide.

A further advantage of the methods is the capability for multipurpose multi-functionalization of beads (added fluorescence and dsDNA binding as examples).

Other advantages include the easy separation of product by simple centrifugation of the membrane-functionalized coated beads, and modular adaptability.

It can be seen that there are many different applications for the membrane-functionalized coated beads benefitting from the added secondary functions and lipid environment. Any function that can be achieved by current commercial beads can be matched by membrane-coated functionalized beads with the added ability of the attached molecules to diffuse about the membrane.

The coated bead constructs with functionalized membranes opens the door to many applications that require fluidity and translational movement for proper or enhanced function. For example, the functional beads and system can be used to study receptor oligomerization, such as G-protein coupled receptors, tumor necrosis receptors and receptors for advanced glycation end-products.

In another example, functionalized lipid-coated beads with the covid spike protein (requiring a membrane environment as SARS CoV2 is an enveloped virus) can be constructed or the host membrane-anchored receptor ACE2 can also be constructed to accurately determine the behavior of these proteins when properly attached to a membrane.

Any hydrophobic fluorophore attached to a short poly-glycine peptide can be inserted into the membrane coating a bead. The resulting bead could then be attached to any hydrophilic polymer of interest that contains the LPETG amino acid sequence, for example. This will allow to create fluorescent beads with added function depending on the hydrophilic polymer.

Signaling cascades can also be studied using this system by anchoring the signal components to the membrane to measure the change in reaction rate when the proteins are anchored. Anchors can be attached to two separate coated beads allowing for 1) forces to be measured between the two membranes by using optical traps; or 2) developing applications that require bead-to-bead binding or recognition.

The system and functionalized beads are particularly suited for biotechnological and medical applications in general and specifically for functions that require or are enhanced by the presence of lipids, lipid bilayers, or characteristics of lipid bilayers such as membrane fluidity, for biologic oligomerization and translational diffusion within the membrane, such as in multivalent adhesion and binding processes inherent to virus infection.

Biosensors in molecular diagnostics, detection, separation, and purification will also be suitable applications of the functionalized constructs.

Functionalized beads are also useful for studies using optical traps. Both force measurements and confocal fluorescence experiments can be used with this system as well.

The technology described herein may be better understood with reference to the accompanying examples, which are intended for purposes of illustration only and should not be construed as in any sense limiting the scope of the technology described herein as defined in the claims appended hereto.

### Example 1

In order to demonstrate the structure and functionality of the lipid-coated constructs, commercial beads of different materials and sizes were coated with a synthetic lipid bilayer and simultaneously functionalized with different hydrophobic peptides. Polystyrene, glass and carboxylate-functionalized beads that were 1 micrometer and 3 micrometer diameters in size were coated and evaluated.

Several different lipid compositions were used to illustrate the methods. The first lipid composition was a 10% (7-nitrobenz-2-oxa-1,3-diazol-4-yl) (NBD), 70% phosphatidylserine (PS) and 20% phosphatidylcholine (PC) mixture. The second lipid composition with a 70% PC and 30% PS mixture was also tested. The (PS) and (PC) lipids were selected because of their presence in the plasma membrane as well as in the membranes of a variety of organelles throughout the cell. NBD is a widely used fluorescent analogue of native membrane lipids.

One preferred hydrophobic molecule that was tested was a transmembrane domain of the protein RAGE with amino acid sequence: GGGGLALGILGGLGTAALLIGVILWRRR (SEQ. ID. NO. 1). The portion of the sequence indicated in bold is designed for transpeptidation. The underlined portion of the sequence was to facilitate solid-phase peptide synthesis and reverse-phase liquid chromatography purification. The peptide was added at a concentration of 1 mg/mL. Other hydrophobic peptides and molecules were also subjected to similar procedures and evaluated.

To demonstrate the multipurpose primary functionalization by coating beads with lipid layers and embedding a hydrophobic peptide, the beads were coated using the methods shown in FIG. 3. The coating methods shown schematically in FIG. 3 represent one preferred embodiment of a simplified protocol for transmembrane peptide insertion or embedding into a synthetic membrane coating. In this example, liposomes were initially formed including transmembrane peptides inserted into the bilayer. Thereafter, the beads were inserted within the interior of the synthetic membrane.

From the flow diagram of FIG. 3, various hydrophobic peptides 32 were mixed with selected lipids or lipid mixtures in the presence of chloroform to form a homogeneous solution. The resulting lipid-peptide mixture was then freeze-dried and subsequently hydrated to produce lipid-peptide suspension. The suspension was sonicated to form hollow liposomes or vesicles with embedded peptides.

The acquired vesicles were then centrifuged (16,973 g for 1 h) and the supernatant was thereafter mixed with beads on low vortex settings for 16 hours. This bead mixture was centrifuged (900 g for 1 min) to pellet the embedded lipid membrane-coated beads, which were then evaluated and tested.

### Example 2

To demonstrate and characterize the capability of secondary functionalization of the primary functionalized beads with hydrophobic molecules, fluorescent bead constructs were made and evaluated.

Turning now to FIG. 4, one type of secondary functionalization of the lipid coated beads was performed to demonstrate the range of possible constructions. FIG. 4 is a schematic representation of secondary functionalization to create fluorescent beads.

As shown in FIG. 4, the method 50 of producing a fluorescent bead as a secondary functionalization of the base functionalized coated bead begins with the bead 52 coated with one or more types of lipids 56 and embedded hydrophobic peptides with an exposed binding site 54. A fluorescent peptide 58 is configured with a linker 62 that can bind with the binding site 54 of the embedded peptide 54 in the lipid coating 56 of the bead 52.

The short peptide 58 used for fluorophore tagging and subsequent transpeptidation with hydrophobic peptide 54 had the following amino acid sequence: RRCGGGSLPETGGG (SEQ. ID. NO. 2). The sequence indicated in bold is designed for transpeptidation. Underlined amino acid “C” is designed for fluorophore tagging via maleimide reaction which was successfully tested with Alexa F 488 and Atto F 488). The peptide was obtained by solid-phase peptide synthesis and purified by reverse-phase liquid chromatography. Other sequences conserving “LPETG” can also be used.

Recombinant sortase A enzyme 60 that was expressed and purified in the laboratory was used for all transpeptidation reactions. Although sortase A is used to illustrate the enzyme, it will be understood that other transpeptidation enzymes and reactions can also be used. The results of the transpeptidation reaction of the fluorescent peptide 58 and the protein binding site 54 of the base bead is a fluorescent bead 64.

Confocal fluorescence microscopy images of beads treated with lipids, the transmembrane domain of the protein RAGE, and a fluorescent peptide linked to this transmembrane domain via sortase, were evaluated. The confocal fluorescence microscopy images of beads treated with lipids and the transmembrane domain of the protein RAGE and after treatment with peptide-Alexa488 and sortase illustrated that the protein inserted in the bead membrane was attached to peptide-Alexa F 488 producing very intense fluorescence.

### Example 3

To further characterize the secondary functionalization capabilities of the primary coated constructs, bead constructs for DNA sensing were created and evaluated. Confocal fluorescence microscopy images of beads showed the successful secondary functionalization with a DNA-binding protein. FIG. 5 is a schematic representation of secondary functionalization 70 to create bead constructs for DNA sensing.

In this illustration, beads 72 coated with lipid layers 74 and embedded peptides 76 with an exposed active coupling site 78 were constructed. A DNA binding protein 80 with a domain or peptide 82 was linked to the peptide 76 domain 78 via a sortase reaction 84. This produced a functionalized bead 86 with a surface layer of exposed DNA binding proteins 80. This secondarily functionalized bead 86 can be used to bind selected DNA 88 as targets bound to the binding proteins 80 of bead 90.

The AIM2 dsDNA binding protein 80 was used to add dsDNA binding functionality to the beads 72 by transpeptidation with the hydrophobic peptide 76 of the following sequence (SEQ. ID. NO. 3):

HHHHHHMKIEEGKLVIWINGDKGYNGLAEVGKKFEKDTGIKVTVEHP
DKLEEKFPQVAATGDGPDIIFWAHDRFGGYAQSGLLAEITPDKAFQD
KLYPFTWDAVRYNGKLIAYPIAVEALSLIYNKDLLPNPPKTWEEIPA
LDKELKAKGKSALMFNLQEPYFTWPLIAADGGYAFKYENGKYDIKDV
GVDNAGAKAGLTFLVDLIKNKHMNADTDYSIAEAAFNKGETAMTING
PWAWSNIDTSKVNYGVTVLPTFKGQPSKPFVGVLSAGINAASPNKEL
AKEFLENYLLTDEGLEAVNKDKPLGAVALKSYEEELVKDPRIAATME
NAQKGEIMPNIPQMSAFWYAVRTAVINAASGRQTVDEALKDAQTENL
YFQGHGSEFMESKYKEILLLTGLDNITDEELDRFKFFLSDEFNIATG
KLHTANRIQVATLMIQNAGAVSAVMKTIRIFQKLNYMLLAKRLQEEK
EKVDKQYKSVTKPKPLSQAEMSPAASAAIRNDVAKQRAAPKVSPHVK
PEQKQMVAQQESIREGFQKRCLPVMVLKAKKPFTFETQEGKQEMFHA
TVATEKEFFFVKVFNTLLKDKFIPKRIIIIARYYRHSGFLEVNSASR
VLDAESDQKVNVPLNIIRKAGETPKINTLQTQPLGTIVNGLFVVQKV
TEKKKNILFDLSDNTGKMEVLGVRNEDTMKCKEGDKVRLTFFTLSKN
GEKLQLTSGVHSTIKVIKAKKKTGGGGSLPETGGG.

This peptide sequence has an amino acid sequence for human AIM2 (SEQ. ID. NO. 4):

MHHHHHHHGSEFMESKYKEILLLTGLDNITDEELDRFKFFLSDEFNI
ATGKLHTANRIQVATLMIQNAGAVSAVMKTIRIFQKLNYMLLAKRLQ
EEKEKVDKQYKSVTKPKPLSQAEMSPAASAAIRNDVAKQRAAPKVSP
HVKPEQKQMVAQQESIREGFQKRCLPVMVLKAKKPFTFETQEGKQEM
FHATVATEKEFFFVKVFNTLLKDKFIPKRIIIIARYYRHSGFLEVNS
ASRVLDAESDQKVNVPLNIIRKAGETPKINTLQTQPLGTIVNGLFVV
QKVTEKKKNILFDLSDNTGKMEVLGVRNEDTMKCKEGDKVRLTFFTL
SKNGEKLQLTSGVHSTIKVIKAKKKTGGGGS.

The peptide also includes a Maltose Binding Protein (MBP) sequence for solubilization (SEQ. ID. NO. 5):

MKIEEGKLVIWINGDKGYNGLAEVGKKFEKDTGIKVTVEHPDKLEEK
FPQVAATGDGPDIIFWAHDRFGGYAQSGLLAEITPDKAFQDKLYPFT
WDAVRYNGKLIAYPIAVEALSLIYNKDLLPNPPKTWEEIPALDKELK
AKGKSALMFNLQEPYFTWPLIAADGGYAFKYENGKYDIKDVGVDNAG
AKAGLTFLVDLIKNKHMNADTDYSIAEAAFNKGETAMTINGPWAWSN
IDTSKVNYGVTVLPTFKGQPSKPFVGVLSAGINAASPNKELAKEFLE
NYLLTDEGLEAVNKDKPLGAVALKSYEEELVKDPRIAATMENAQKGE
IMPNIPQMSAFWYAVRTAVINAASGRQTVDEALKDAQT

This peptide sequence also has a TEV protease recognition site (ENLYFQG) (SEQ. ID. NO. 6) to remove MBP and a recognition site 78 for transpeptidation (LPETG) (SEQ. ID. NO. 7).

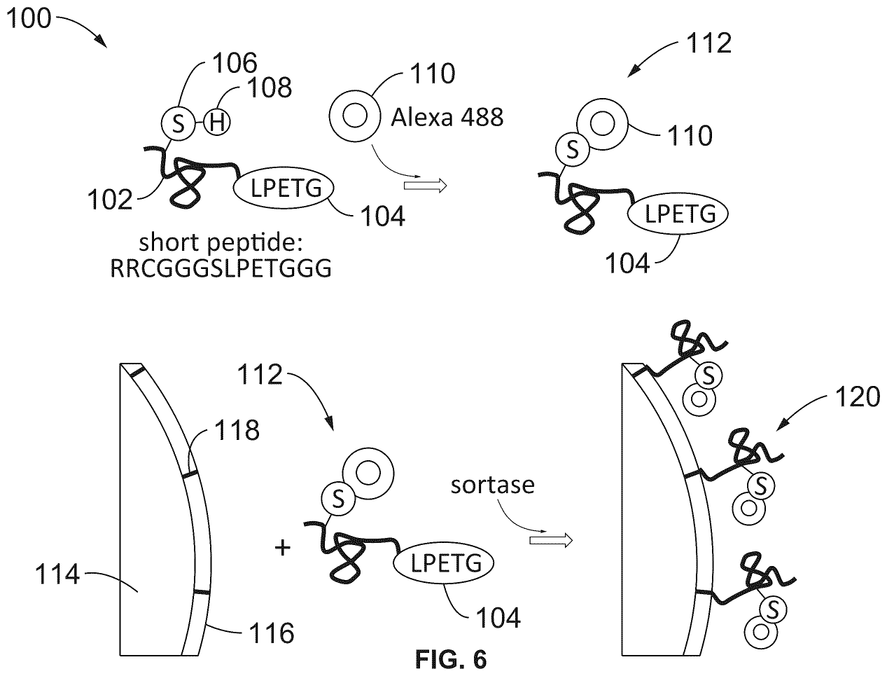

The insertion of transmembrane peptide in membrane-coated beads and transpeptidation with a fluorescent-labeled peptide was verified 100 as shown in FIG. 6. Initially, a short peptide 102 with a transpeptidation sequence 104 at one end and sulfur 106 and hydrogen 108 groups at the other end, which were used for fluorophore tagging. In this embodiment, the (Alexa F 488) fluorescent molecule 110 is bound to the peptide 102 by the maleimide reaction to produce a fluorescent-labeled peptide 112 for binding with a membrane-bound hydrophobic anchor protein 118 embedded into the lipid layer coating 116 of a bead 114. The Alexa F 488-labeled peptide 112 was attached to the anchor peptide 118 by sortase-mediated transpeptidation of the inserted transmembrane anchor peptide 118. The secondary functionalization of the bead 114 with the labeled short peptide 112 produced a bead with a labeled surface 120. The binding of the peptide 112 with the anchor peptide 118 of the bead 114 was verified with two-dimensional confocal microscopy scans.

From the description herein, it will be appreciated that the present disclosure encompasses multiple implementations of the technology which include, but are not limited to, the following:

A functionalized bead construct, comprising one or more micrometer-scaled beads, the beads having an outer surface; a primary functionalization of a lipid coating on the outer surface of the beads; and a secondary functionalization of a biologic on the lipid coating of the beads.

The construct of any preceding or following implementation, wherein the bead is made of a material selected from the group of materials consisting of polystyrene, glass, plastic, magnetic particles, metal particles and carboxylate-functionalized polystyrene.

The construct of any preceding or following implementation, wherein the coating of the bead comprises a lipid bi-layer coating.

The construct of any preceding or following implementation, wherein the lipid coating comprises a coating of a plurality of lipids selected from the group comprising (7-nitrobenz-2-oxa-1,3-diazol-4-yl) (NBD), 70% phosphatidylserine (PS) and 20% phosphatidylcholine (PC).

The construct of any preceding or following implementation, further comprising: an anchor peptide embedded in the lipid layer; wherein the anchor peptide couples to the secondary functionalization biologic.

The construct of any preceding or following implementation, wherein the anchor peptide is a transmembrane peptide.

The construct of any preceding or following implementation, wherein the anchor peptide has an amino acid sequence comprising (SEQ. ID. NO. 1).

The construct of any preceding or following implementation, wherein the anchor peptide has an amino acid sequence comprising (SEQ. ID. NO. 2).

The construct of any preceding or following implementation, wherein the anchor peptide has an amino acid sequence comprising (SEQ. ID. NO. 3).

The construct of any preceding or following implementation, wherein the biologic is a material selected from the group of a peptide, a protein, a nucleic acid, DNA-binding protein, RNA-binding protein, and a fluorescent peptide.

The construct of any preceding or following implementation, wherein the biologic is a material selected from the group consisting of antibodies, collagen, protein L, protein G, protein BSA, streptavidin, biotin, and neutravidin.

The construct of any preceding or following implementation, wherein the biologic peptide further comprises a fluorescent molecule.

A method for fabricating a functionalized bead construct, the method comprising: providing one or more micrometer scaled beads, the beads having an outer surface; functionalizing the beads with a primary functionalization of a coating of the outer surface of the beads with a lipid coating; and functionalizing the beads with a secondary functionalization of at least one biologic to produce a functionalized bead construct.

The method of any preceding or following implementation, further comprising simultaneously coating the bead with a plurality of lipids and anchor peptides.

The method of any preceding or following implementation, wherein the coating of the bead comprises a lipid bi-layer coating.

A method for fabricating a functionalized bead construct, the method comprising: (a) forming a plurality of lipids and anchor peptides to produce peptide embedded liposomes; (b) providing one or more micrometer scaled beads, the beads having an outer surface; (c) mixing the beads with the liposomes to produce a coating of the outer surface of the beads with a protein-embedded lipid coating; and (d functionalizing the beads with a secondary functionalization of at least one biologic to produce a functionalized bead construct.

The method of any preceding or following implementation, wherein the coating of the bead comprises a lipid bi-layer coating.

The method of any preceding or following implementation, wherein the anchor peptide is a transmembrane peptide.

The method of any preceding or following implementation, wherein the anchor peptide has an amino acid sequence selected from the group of (SEQ. ID. NO. 1), (SEQ. ID. NO. 2) and (SEQ. ID. NO. 3).

The construct of any preceding or following implementation, wherein the biologic is a material selected from the group of a peptide, a protein, a nucleic acid, DNA-binding protein, RNA-binding protein, and a fluorescent peptide.

As used herein, the term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these groups of elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system, or method.

As used herein, the term “set” refers to a collection of one or more objects. Thus, for example, a set of objects can include a single object or multiple objects.

Relational terms such as first and second, top and bottom, upper and lower, left and right, and the like, may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, apparatus, or system, that comprises, has, includes, or contains a list of elements does not include only those elements but may include other elements not expressly listed or inherent to such process, method, article, apparatus, or system. An element proceeded by “comprises . . . a”, “has . . . a”, “includes . . . a”, “contains . . . a” does not, without more constraints, preclude the existence of additional identical elements in the process, method, article, apparatus, or system, that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than equal to +10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way, but may also be configured in ways that are not listed.

Benefits, advantages, solutions to problems, and any element(s) that may cause any benefit, advantage, or solution to occur or become more pronounced are not to be construed as a critical, required, or essential feature or element of the technology described herein or any or all the claims.

In addition, in the foregoing disclosure various features may be grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after the application is filed. Accordingly, the reader should consult the application as filed for the original content of the disclosure. Any deletion of content of the disclosure should not be construed as a disclaimer, forfeiture, or dedication to the public of any subject matter of the application as originally filed.

The following claims are hereby incorporated into the disclosure, with each claim standing on its own as a separately claimed subject matter.

Although the description herein contains many details, these should not be construed as limiting the scope of the disclosure, but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A functionalized bead construct, comprising:
(a) one or more micrometer-scaled beads, said beads having an outer surface;
(b) a primary functionalization of a lipid coating on said outer surface of said beads; and
(c) a secondary functionalization of a biologic on said lipid coating of said beads.

2. The construct of claim 1, wherein said bead is made of a material selected from the group of materials consisting of polystyrene, glass, plastic, magnetic particles, metal particles and carboxylate-functionalized polystyrene.

3. The construct of claim 1, wherein said coating of said bead comprises a lipid bi-layer coating.

4. The construct of claim 1, wherein said lipid coating comprises a coating of a plurality of lipids selected from the group comprising 10% (7-nitrobenz-2-oxa-1,3-diazol-4-yl) (NBD), 70% phosphatidylserine (PS) and 20% phosphatidylcholine (PC).

5. The construct of claim 1, wherein said biologic is a material selected from the group of a peptide, a protein, a nucleic acid, DNA-binding protein, RNA-binding protein, and a fluorescent peptide.

6. The construct of claim 1, wherein said biologic is a material selected from the group of a peptide, a protein, a nucleic acid, DNA-binding protein, RNA-binding protein, and a fluorescent peptide.

7. The construct of claim 1, wherein said biologic is a material selected from the group consisting of antibodies, collagen, protein L, protein G, protein BSA, streptavidin, biotin, and neutravidin.

8. The construct of claim 1, further comprising:
an anchor peptide embedded in said lipid layer;
wherein said anchor peptide couples to said secondary functionalization biologic.

9. The construct of claim 8, wherein said anchor peptide is a transmembrane peptide.

10. The construct of claim 8, wherein said anchor peptide has an amino acid sequence comprising (SEQ. ID. NO. 1).

11. The construct of claim 8, wherein said anchor peptide has an amino acid sequence comprising (SEQ. ID. NO. 2).

12. The construct of claim 8, wherein said anchor peptide has an amino acid sequence comprising (SEQ. ID. NO. 3).

13. The construct of claim 8, wherein said biologic peptide further comprises a fluorescent molecule.

14. The construct of claim 8, wherein said anchor peptide is a transmembrane peptide.

15. A method for fabricating a functionalized bead construct, the method comprising:
(a) providing one or more micrometer scaled beads, said beads having an outer surface;
(b) functionalizing the beads with a primary functionalization of a coating of said outer surface of said beads with a lipid coating; and
(c) functionalizing the beads with a secondary functionalization of at least one biologic to produce a functionalized bead construct.

16. The method of claim 15, further comprising:
simultaneously coating said bead with a plurality of lipids and anchor peptides.

17. The method of claim 15, wherein said coating of said bead comprises a lipid bi-layer coating.

18. A method for fabricating a functionalized bead construct, the method comprising:
(a) forming a plurality of lipids and anchor peptides to produce peptide embedded liposomes;
(b) providing one or more micrometer scaled beads, said beads having an outer surface;
(c) mixing said beads with said liposomes to produce a coating of said outer surface of said beads with a protein-embedded lipid coating; and
(d) functionalizing the beads with a secondary functionalization of at least one biologic to produce a functionalized bead construct.

19. The method of claim 18, wherein said coating of said bead comprises a lipid bi-layer coating.

20. The method of claim 18, wherein said anchor peptide has an amino acid sequence selected from the group of (SEQ. ID. NO. 1), (SEQ. ID. NO. 2) and (SEQ. ID. NO. 3).


SEQUENCE LISTING (7 sequences)

SEQ ID NO: 1 - protein, 28 aa, synthetic construct
GGGGLALGILGGLGTAALLIGVILWRRR

SEQ ID NO: 2 - protein, 14 aa, synthetic construct
RRCGGGSLPETGGG

SEQ ID NO: 3 - protein, 739 aa, synthetic construct
MHHHHHHMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTENLYFQGHGSEFMESKYKEILLLTGLDNITDEELDRFKFFLSDEFNIATGLHTANRIQVATLMIQNAGAVSAVMKTIRIFQKLNYMLLAKRLQEEKEKVDQYKSVTKPKPLSQAEMSPAASAAIRNDVAKQRAAPKVSPHVKPEQKQMVAQQESIREGFQKRCLPVMVLKAKKPFTFETQEGKQEMFHATVATEKEFFFVKVFNTLLKDKFIPKRIIIIARYYRHSGFLEVNSASRVLDAESDQKVNVPLNIIRKAGETPKINTLQTQPLGTIVNGLFVVQKVTEKKKNILFDLSDNTGKMEVLGVRNEDTMKCKEGDKVRLTFFTLSKNGEKLQLTSGVHTSIKVIKAKKKTGGGGSLPETGGG

SEQ ID NO: 4 - protein, 359 aa, synthetic construct
MHHHHHHGSEFMESKYKEILLLTLGDNITDEELDRFKFFLSDEFNIATGKLHTANRIQVATLMIQNAGAVSAVMKTIRIFQKLNYMLLAKRLQEEKEKVDKQYKSVTKPKPLSQAEMSPAASAAIRNDVAKQRAAPKVSPHVKPEQKQMVAQQESIREWGFQKRCLPVMVLKAKKPFTFETQEGKQEMFHATVATEKEFFFVKVFNTLLKDKFIPKRIIIIARYYRHSGFLEVNSASRVLDAESDQKVNVLPNIIRKAGETPKINTLQTQPLGTIVNGLFVVQKVTEKKKNILFDLSDNTGKMEVLGVRNEDTMKCKEGDKVRLTFFTLSKNGEKLQLTSGVHSTIKVIKAKKKTGGGS

SEQ ID NO: 5 - protein, 364 aa, synthetic construct
MKIEEGKLVIWINGDKYNGLAEVGKKFEKDTGIKVTVEHDPKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPFVGVLSAGINAASPNKELAKEFLENYLLTDEGLEAVNKDKPLGAVAALKSYEEELVKDPRIAATMENAQKGEIMPNIPQMSAFWYAVRTAVINAASGRQTVDEALKDAQT

SEQ ID NO: 6 - protein, 7 aa, synthetic construct
ENLYFQG

SEQ ID NO: 7 - protein, 5 aa, synthetic construct
LPETG